(19) United States
(12) Patent Application Publication
(10) Pub. No.: US 2020/0121402 A1
(43) Pub. Date: Apr. 23, 2020

(54) ACTIVE SYSTEM FOR ROBOTIC SURGERY

(71) Applicant: Luiz Lanat Pedreira de Cerqueira Filho, Orlando, FL (US)

(72) Inventor: Luiz Lanat Pedreira de Cerqueira Filho, Orlando, FL (US)

(21) Appl. No.: 16/578,392

(22) Filed: Sep. 22, 2019

Related U.S. Application Data
(60) Provisional application No. 62/747,636, filed on Oct. 18, 2018.

(51) Int. Cl. A61B 34/30

(52) U.S. Cl. CPC A61B 34/30; A61B 2034/2057

(57) ABSTRACT

A method for robotic active surgery, comprising the steps of, (a) get an image of a surgical site, (b) select in said image an object to track, and (d) accompanying a movement of said object with at least one active robotic instrument is provided. A method for a robotic active surgery, including the steps of, selecting on a screen at least one point in a surgical site image, choosing in a system database the best technique to perform said procedure, and using an active robotic instrument to perform said procedure is provided. A method further including selectively switching the operating mode of said active robotic instrument between an active mode and a passive mode is provided.

This application claims an invention which was disclosed in provisional application No. 62/735,165, filed Sep. 23, 2018 entitled “Active System for Robotic Cardiac Surgery and method”, and in provisional application No. 62/747,636, filed Oct. 18, 2018 entitled “Active System for Robotic Cardiac Surgery and method”, and in. The benefit under 35 USC § 119(e) of the United States provisional application is hereby claimed, and the afore mentioned application is hereby incorporated herein by reference.

### FIELD OF INVENTION

The invention is in the field of medical devices for performing robotic surgery and pertains particularly to an active system for robotic surgery made to performing robotic surgery in a moving tissue and methods of use.

### BACKGROUND

In the art of medical devices for performing robotic surgery many different types of systems for robotic surgery have been developed. In prior art, to perform the suture in a beating heart it is difficult because the tissue constantly changes place with the contraction of the heart. One problem with the traditional robotic surgery, it is necessary that the tissue is not moving or stabilized for the operator perform the suture. Another limitation of the current devices, suturing a contracting heart is difficult because the operator observes a moving heart, the place of the suture is seen constantly changing place with the heart beats, it makes the surgery difficult, demand longer time, and incrise the complications.

Another limitation of the robotic surgery is the need to stabilize and minimize the movement of selected areas of the tissue or stop a heart pulsation during surgical procedures.

Current robotic surgery devices for robotic surgery such as conventional cardiac instrumental, video instruments, robotic instruments and others the surgery systems, needs to be performed with the heart in asystole with the patient on extracorporeal circulation or with the use of the tissue stabilizer.

It has not been reported aa active system for robotic surgery on a moving tissue, with the operator performing the surgery observing the tissue as if standing still. The possibility of performing a technically perfect surgery in a heart beating as it is standing still is an important development in robotic surgery.

Therefore, what is clearly needed is an active system for robotic surgery made to performing robotic surgery in a moving tissue and methods of use that solves the problems mentioned above.

### SUMMARY OF THE INVENTION

In one embodiment the invention a method for robotic active surgery, comprising the steps of, (a) get an image of a surgical site, (b) select in said image an object to track, and (d) accompanying a movement of said object with at least one active robotic instrument is provided.

Also, in other embodiment the invention further including using said selection to scan every frame looking for said object.

Also, in other embodiment the invention further including improving tracking performance during tracking.

Also, in other embodiment the invention further including determining said object locations and changes of said object.

Also, in other embodiment the invention further including learning how said objects looks like and use that information to scan every frame searching for said object.

Also, in other embodiment the invention further including defining said object location in relation to at least one camera head.

Also, in other embodiment the invention further including defining a working distance of said camera head relative to said object.

Also, in other embodiment the invention further including accompanying with at least one active camera head the movement of said object in space.

Also, in other embodiment the invention further including accompanying with at least one active robotic instrument said movement of said object in said surgery site.

Also, in other embodiment the invention further including transmitting said image to a screen.

Also, in other embodiment the invention further including observing in a screen said image of said moving object as if it is standing still.

Also, in other embodiment the invention further including choosing in a toolbox a tip tool for said robotic active robotic instrument inside said surgical site.

Also, in other embodiment the invention further including changing said robotic active instrument tip tool inside said surgical site.

Also, in other embodiment the invention further including using at least one active robotic instrument to perform said surgery.

Also, in other embodiment the invention further including sensing movement of said object and generating sensor output signals in response thereto;

Also, in other embodiment the invention further including calculating a demanded force signal for at least one actuator in response to said sensor output signals.

Also, in other embodiment the invention further including regulating a damped resistance to movement between said active robotic instrument and said object when said demanded force signal is within a predetermined range.

In another embodiment the invention including a method for a robotic active surgery, including the steps of, selecting on a screen at least one point in a surgical site image, choosing in a system database the best technique to perform said procedure, and using a robotic active robotic instrument to perform said procedure.

In another embodiment the invention including a method for robotic active surgery, the method being characterized by, detecting an event, and moving a position of an ative robotic instrument in response to said detected event.

Also, in other embodiment the method further including selectively switching the operating mode of said active robotic instrument between an active mode and a passive mode.

Also, in other embodiment the method including a user selection.

### BRIEF DESCRIPTION OF THE DRAWINGS

FIG. 1 is a cross-sectional view of the active system for robotic surgery, according to another embodiment of the invention.

FIG. 2 is a front view of a camera 5B with three heard 7, according to another embodiment of the invention.

FIG. 3 is a side view of an active robotic instrument 3 transfixing a wall 6A, the active robotic instrument 3 is connected in a robotic arm 2, according to another embodiment of the invention.

FIG. 4 is a front view of 3D robotic camera 5B with a head 7A and a head 7B. Each heard 7 captures an image 12 of the selected object 1, according to another embodiment of the invention.

FIG. 5 is a front view of the active robotic camera that captures the image 12 of the object 1, according to another embodiment of the invention.

### DETAILED DESCRIPTION OF THE PREFERRED EMBODIMENTS

The inventor provides an active system for robotic surgery made to performing robotic surgery in a moving tissue and methods of use. One characteristic of some embodiments is an active system of coordinated movement of surgical instruments to perform cardiac surgery with a heart 11 in motion. Another characteristic of some embodiments of the invention, the instruments involved in the surgery is adapted to actively tracking the movement of a selected object 1 in the tissue, including the camera 5B. Another characteristic of some embodiments of the invention, the operator observes on a screen 13 (it is not draw) the object 1 that is in motion as if it is stopped. Another characteristic of some embodiments of the invention, the invention functions substantially similarly to the active suspension employed in cars in which the suspension is actively moved relative to the ground and the chassis of the car remains stable.

The invention is described in enabling detail in the following examples, which may be more than one embodiment of the invention, together with the accompanying drawings in which like numerals re similar components. Additionally, the structures described herein can be embodied as integrated components or as separate components.

Another characteristic of some embodiments of the invention, the operator has control of the robotic surgical instruments and the camera 5B, however the instruments is adapted to independently and actively follow the movement of the observed tissues. Another characteristic of some embodiments of the invention, the system is adapted to be enabled or disabled during the procedure. Another characteristic of some embodiments of the invention, the operator choose which active robotic instruments will have active movement or work in passive mode. For example, the operator initiates robotic surgery in passive mode, upon encountering the object 1 having movement as the cardiac tissue, the operator select the object 1 and activates the active mode, the selected tissue is tracked, the object 1 marked and observed by the operator is seen without motion in the screen 13. Another characteristic of some embodiments of the invention, the operator performs the surgery accurately on the heart 11 that beats normally as it is standing still. Another characteristic of some embodiments of the invention, the operator also use two or more camera 5B, the camera 5B that observes the moving object 1, and another that follow the movement of the object 1 that is seen stopped, the operator observe the area operated on two or more screen 13.

Another characteristic of some embodiments of the invention, the operator manipulates the camera 5B, determines the working distance and the active robotic instruments autonomously accompany the movement of the selected object 1 on the tissue. Another characteristic of some embodiments of the invention, the operator performs an anastomosis in the heart 11 that knocks by observing the image 12 on the screen 13 as if it is standing still.

Another characteristic of some embodiments of the invention, the instruments have active joints and buffers with sensors that allow the operator to actively follow the movement of the object 1 in the tissue with 3D camera 5B, usually with night vision.

Another characteristic of some embodiments of the invention, each active robotic instrument 3 has sensors, these sensors measure distance and pressure. Another characteristic of some embodiments of the invention, the suture is performed by the operator with a needle holder 5A or the system use an automatic suture tip tool 5. Another characteristic of some embodiments of the invention, the system has 3D camera 5B, night vision system that continually scans in detail the entire area that will be operated.

Another characteristic of some embodiments of the invention, there are more than one way to mark the object 1 as the tissue, for example: use a tracker system substantially similar to https://youtu.be/1GhNXHCQGsM, or use of flowering placed on the tissue, or capture the image 12 of the area and mark the object 1 on the image 12, but are not limited to them. Another characteristic of some embodiments of the invention, the use of a second the camera 5B and or a third the camera 5B aid in the accuracy of the object 1 location. Another characteristic of some embodiments of the invention, a laser beam on the camera 5B illuminate the object 1, thus designating the object 1 for the second camera 5B to follow.

Another characteristic of some embodiments of the invention, in the case of fluorescent object 1, a fluorescent ink is used, and the 3D camera 5B has a night vision system and interface that accurately identifies the dots of flowing ink, one of tip tool 5 puts a dot of flowing paint on the tissue at the desired location. In this example, the camera 5B starts to screen 13 the blossoming object 1, and the operator watches still the image 12 of the object 1 on the screen 13. In this example, the operator marks new object 1 in the workplace, the operator changes from one object 1 to the other object 1 with an aiming sight. In this example, the operator should keep the object 1 he wants stopped within the aiming sight, according to another embodiment of the invention.

Another characteristic of some embodiments of the invention, in case of capturing the image 12 of the area where the object 1 is, the operator first captures the image 12 of the area where the object 1 is and then marks the desired object 1 in the image 12. In this example, the marked object 1 is in the tissue, via the interface, it works substantially similar to a person's face market on modern cell phones. In this example, when the tissue moves the new image 12 is compared with the marked image 12. In this example, the camera 5B is automatically repositioned to the new object 1 position and the other selected instruments are also automatically repositioned. In this example, the use of a second the camera 5B without active movement relative to the object 1 greatly aid in its location.

Another characteristic of some embodiments of the invention, in case of use a tracker system that learn, it is much easier to mark the desired object 1 on tissue. Another characteristic of some embodiments of the invention, the operator selects the desired suture line so that a robotic arm 2 is adapted to automatically suture with a suture tool or is adapted to manipulate the robotic arm 2 to suture with a needle holder 5A. In this example, the operator selecting on the screen 13 at least one point in a surgical site 10 image 12; choosing in a system database the best technique to perform said procedure; and using a robotic active robotic instrument 3 to perform said procedure. Another characteristic of some embodiments of the invention, the object 1 of observation is selected by the operator. Another characteristic of some embodiments of the invention, most important of all is the stable sight the operator gets from the observed object 1.

Another characteristic of some embodiments of the invention, the system is adapted to track an object 1 in the image 12. Another characteristic of some embodiments of the invention, a certain part of the image 12 is selected, and this part is accompanied by a frame. Another characteristic of some embodiments of the invention, the invention uses this principle for the operator to mark the object 1 and choose the working distance. Another characteristic of some embodiments of the invention, the camera 5B accompanies the movement of the object 1. Another characteristic of some embodiments of the invention, once the object 1 is marked the camera 5B starts to follow the movement of the object 1, so that the object 1 seems stopped for those who watch on the screen 13, according to another embodiment of the invention.

Another characteristic of some embodiments, the invention utilizes sensors for data collection and this information identifies the movement of an object 1 of the tissue to indicate which types of movement are to be employed by the active robotic instrument 3 cluster. In this example, the articulations of the instruments move independently for each instrument 3, minimizing or canceling the movement vision of the observed object 1.

Another characteristic of some embodiments of the invention, the surgery with the invention is adapted to be performed in two ways: the camera 5B and at least one of the instruments involved in the surgery actively accompany the movement of the observation object 1 (appear to be stopped by the operator), or only the camera 5B actively follows the observed object 1 (only the observed object 1 seems stopped), but is not limited to them.

Another characteristic of some embodiments of the invention, in the first case, when the instruments are involved in the surgery follow the movement of the selected object 1, the operator has the image 12 on his screen 13 as if the tissue were stopped even though the heart 11 is beating normally, the instruments also appear stopped on the operator's screen 13 because all the set moves in a harmonic manner relative to the working object 1. Another characteristic of some embodiments of the invention, this is extremely important for the operator to perform the surgery. Another characteristic of some embodiments of the invention, in such a situation, where the heart 11 is beating more the observed image 12 is stopped, the operator has the vision necessary to perform a suture by himself moving the handle of the instruments having their movements corrected by the invention. Another characteristic of some embodiments of the invention, the invention is adapted to correct the view of tissue movement relative to the camera 5B and instruments efficiently, improving the operator's ability to operate on moving object 1. Another characteristic of some embodiments of the invention, the operator accompanies both the robotic arm 2 and the target.

Another characteristic of some embodiments of the invention, in the second case in which only the camera 5B actively follows the observed object 1, the system must be pre-programmed to perform the suture, the operator will only inform, with the aiming screen 13 that observes a stopped object 1, where it must be sutured. Another characteristic of some embodiments of the invention, the operator selects a sequence of objects 1 from a curve or the first and last object 1 of a line. Another characteristic of some embodiments of the invention, the system's control architecture allows the operator to select and track incisions and stitches. Another characteristic of some embodiments of the invention, a tip tool 5 automatic suture is attached to the robotic arm 2 and performs the sutures in automatic mode, the system automatically calculates equally spaced objects 1 based on a suture stitch contour. Another characteristic of some embodiments of the invention, the system is adapted to be programmed to perform various surgical techniques, analyze the circumstances and choose the most appropriate suture technique in its database, the operator determines the location and authorizes the suture, but the system performs the movements quickly and autonomously. Another characteristic of some embodiments of the invention, the system has supervised autonomy. Another characteristic of some embodiments of the invention, the operator follows the target with the camera 5B, which guides the active robotic instruments.

Another characteristic of some embodiments of the invention, a system that guides the movement of one or more robotic surgical instruments to actively follow an object 1 that moves on the surface of the tissue.

The preferred embodiments according to the invention are shown in FIGS. 1-5

FIG. 1 is a cross-sectional view of the active system for robotic surgery, according to another embodiment of the invention. A in this example the wall 6A and a sternum bone 6B is seen. In this example two active robotic instrument 3 are transfixing the wall 6A. Another characteristic of this embodiments of the invention, the active robotic instrument 3B in the left side L is using a camera 5B and the other active robotic instrument 3A in the right left side R is using a needle holder 5A. Another characteristic of this embodiments of the invention, an object 1 is the selected tissue area being observed by camera 5B on the surface of the pulsing heart 11. In this example the object 1 moves continuously from position 1A to position 1B. In this example the object 1 is always moving from the position 1A to the position 1B. In some embodiments the object 1 is standing still. Another characteristic of this embodiments of the invention, the camera 5B is embedded in an articulated active adapter 4B that has active system capable of movement in the cartesian axes X, Y, Z. Another characteristic of this embodiments of the invention, the needle holder 5A is embedded in an articulated active adapter 4A that has active system capable of movement in the cartesian axes X, Y, Z. Each adapter 4 is engaged in an active robotic instrument 3. Another characteristic of this embodiments of the invention, the active robotic instrument 3A is fitted into a robotic arm 2A and the active robotic instrument 3B is fitted into a robotic arm 2B. Another characteristic of this embodiments of the invention, the operator selectively switching the operating mode of eatch active robotic instrument 3 between an active mode and a passive mode.

Another characteristic of this embodiments of the invention, when the active mode is selected, the camera 5B perfectly follows the movement of the object 1 from position 1A to position 1B, and the operator observes in the screen 13 the object 1 as if it is stopped. Another characteristic of this embodiments of the invention, the system also tracks the movement of object 1 so that the operator observes the needle holder 5A on the screen 13 as if standing still in relation to object 1, the needle holder 5A also follows the movement of the object 1 from position 1A to position 1B. Another characteristic of this embodiments of the invention, the invention enables the operator to perform a perfect surgery even with the beating heart 11. Another characteristic of this embodiments of the invention, the needle holder 5A is adapted to be replaced by a tip claw 5C, a tip scissors 5D or other tip tool 5 during the procedure. Another characteristic of this embodiments of the invention, the tips tool 5 are stored during the procedure in the toolbox 5E in the surgical site 10.

Another characteristic of some embodiments of the invention, the invention also functions in a conventional robotic instrument 3 adapted to select and follow the object 1 movement, according to another embodiment of the invention.

Another characteristic of some embodiments of the invention, to perform the surgical procedure in a selected object 1, the operator informs the system which procedure to perform, for example, a suture, a cut but is not limited to them. Another characteristic of some embodiments of the invention, the operator selects in the screen 13 where to perform the procedure.

Another characteristic of some embodiments of the invention, the operator chooses in the toolbox 5E the best tip tool 5 for the procedure, the system chooses in its database the best technique to perform the procedure with the chosen tip tool 5. Another characteristic of some embodiments of the invention, during the procedure the operator use the toolbox 5E to change the tip tool 5, inside the cavity, according to another embodiment of the invention.

Another characteristic of some embodiments of the invention, a method for robotic active surgery, comprising the steps of: (a) get the image 12 of the surgical site 10; (b) select in said image 12 an object 1 to track; and (d) accompanying a movement of said object 1 with at least one active robotic surgical instrument 3. Another characteristic of some embodiments of the invention, the method further comprising using said selection to scan every frame looking for said object 1. Another characteristic of some embodiments of the invention, the method further comprising improving the tracking performance during tracking. Another characteristic of some embodiments of the invention, the method further comprising determining said object 1 locations and changes of said object 1. Another characteristic of some embodiments of the invention, the method further comprising learning how said objects 1 looks like and use that information to scan every frame searching for said object 1. Another characteristic of some embodiments of the invention, the method further comprising defining said object 1 location in relation to at least one camera 5B the head 7. Another characteristic of some embodiments of the invention, the method further comprising defining a working distance of said camera 5B the head 7 relative to said object 1. Another characteristic of some embodiments of the invention, the method further comprising accompanying with at least one camera 5B the head 7 the movement of said object 1 in space. Another characteristic of some embodiments of the invention, the method further comprising accompanying with at least one robotic active robotic instrument 3 said movement of said object 1 in said surgery site 10. Another characteristic of some embodiments of the invention, the method further comprising transmitting said image 12 to the screen 13. Another characteristic of some embodiments of the invention, the method further comprising observing in the screen 13 said image 12 of said moving object 1 as if it is standing still. Another characteristic of some embodiments of the invention, the method further comprising, choosing in a toolbox a tip tool 5 for said robotic active robotic instrument 3 inside said surgical site 10. Another characteristic of some embodiments of the invention, the method further comprising changing said robotic active robotic instrument 3 tip tool 5 inside said surgical site 10. Another characteristic of some embodiments of the invention, the method further comprising using at least one active robotic instrument 3 to perform said surgery. Another characteristic of some embodiments of the invention, the method further comprising sensing movement of said object 1 and generating sensor output signals in response thereto. Another characteristic of some embodiments of the invention, the method further comprising calculating a demanded force signal for at least one actuator in response to said sensor output signals. Another characteristic of some embodiments of the invention, the method further comprising regulating a damped resistance to movement between said active robotic instrument 3 and said object 1 when said demanded force signal is within a predetermined range.

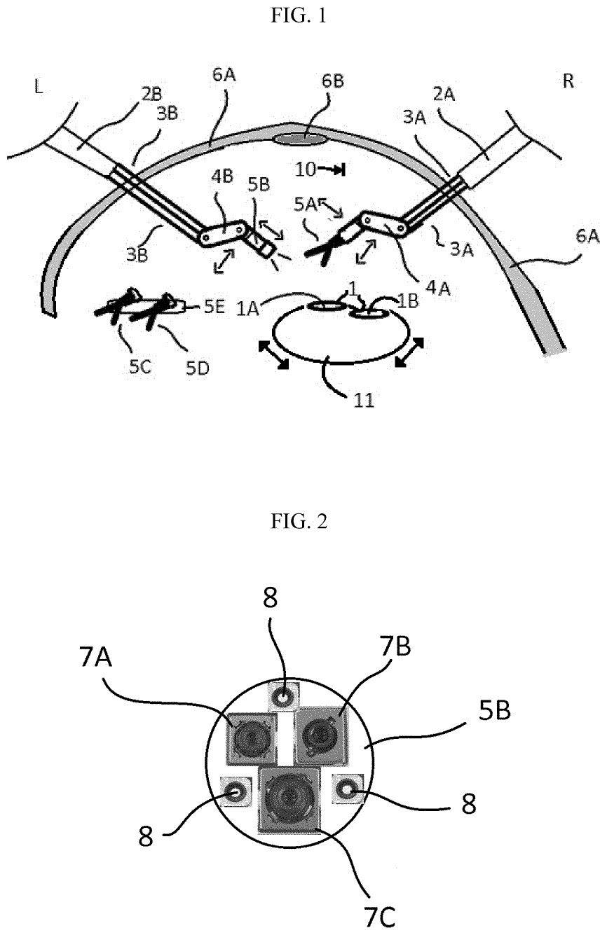

FIG. 2 is a front view of a camera 5B with three heard 7, according to another embodiment of the invention. Another characteristic of this embodiments of the invention, the heard 7A captures the image 12 of the selected object 1, the heard 7B captures the image 12 of the selected object 1, the heard 7C captures the image 12 of the selected object 1. Another characteristic of this embodiments of the invention, this increases the image 12 quality and increases the accuracy of the location of the selected object 1 to perform the surgery. Another characteristic of this embodiments of the invention, the cartesian points X, Y and Z are located by the system using the three images, the system compares the images to locate the object 1, according to another embodiment of the invention. Another characteristic of this embodiments of the invention, a led 8 is used to illuminate the surgical site 10, according to another embodiment of the invention.

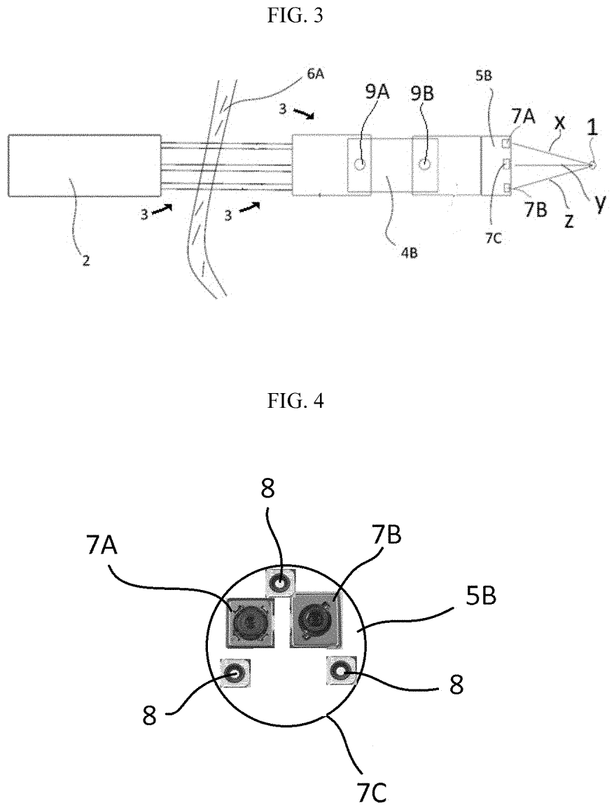

FIG. 3 is a side view of an active robotic instrument 3 transfixing the wall 6A, according to another embodiment of the invention. Another characteristic of this embodiments of the invention, the active robotic instrument 3 is connected in the robotic arm 2. Another characteristic of this embodiments of the invention, the active adapter 4B has a joint 9A, and a joints 9B that moves actively. Another characteristic of this embodiments of the invention, the camera 5B has the heard 7A, the heard 7B, the heard 7C. The heard 7A observes object 1 by the axis view X, the heard 7B observes object 1 by the axis of view Z and the heard 7C observes object 1 by the axis of view Y. Another characteristic of this embodiments of the invention, the images obtained on each axis accurately locate object 1 by the system, according to another embodiment of the invention. Another characteristic of this embodiments of the invention, a method for a robotic active surgery, comprising the steps of: selecting on the screen 13 at least one point in the surgical site 10 image; choosing in a system database the best technique to perform said procedure; and using a robotic active surgery instrument 3 to perform said procedure,

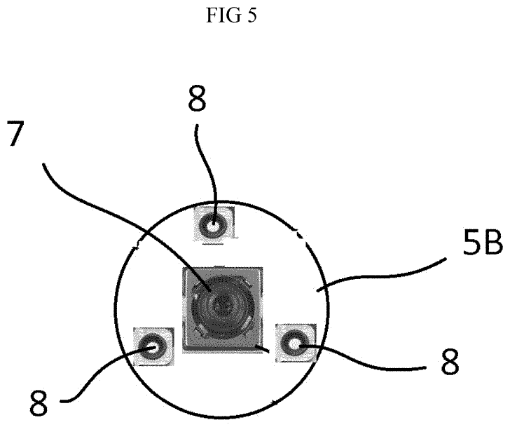

FIG. 4 is a front view of a 3D robotic camera 5B with the head 7A and head 7B, according to another embodiment of the invention. Another characteristic of this embodiments of the invention, each heard 7 captures the image 12 of the selected object 1. Another characteristic of this embodiments of the invention, two heads provide 3D image 12, there is better accuracy of the location of the object 1 selected for performing the surgery than the camera 5B with one heard 7. Another characteristic of this embodiments of the invention, the cartesian object 1 axis X, Y and Z are located by the heart 7 and compared.

Another characteristic of some embodiments of the invention, a method for robotic active surgery, the method being characterized by detecting an event; and moving a position of an active robotic surgery instrument 3 in response to said detected event. Another characteristic of some embodiments of the invention, the method according further comprising selectively switching the operating mode of said robotic active instrument 3 between an active mode and a passive mode. Another characteristic of some embodiments of the invention, the method wherein said event comprises a user selection.

FIG. 5 is a front view of the active robotic camera 5B that captures the image 12 of the object 1, according to another embodiment of the invention. Another characteristic of this embodiments of the invention, the image 12 of the selected object 1 is captured and tracking by the system. Another characteristic of some embodiments of the invention, the operator selects a sequence of points in the screen 13 where the surgical procedure will be made.

It will be apparent to one skilled in the art that the invention may be provided with some or all of the mentioned features and components without departing from the spirit and scope of the invention. For purposes of comparing various embodiments, certain aspects and advantages of these embodiments are described. Not necessarily all such aspects or advantages are achieved by any particular embodiment. Thus, for example, various embodiments can be carried out in a manner that achieves or optimizes one advantage or group of advantages as described herein without necessarily achieving other aspects or advantages as can also be described or suggested herein. It will also be apparent to the skilled artisan that the embodiments described above are specific examples of a broader invention which may have greater scope than any of the singular descriptions taught. There may be many alterations made in the descriptions without departing from the spirit scope of the invention.

Although certain preferred embodiments and examples are disclosed, inventive subject matter extends beyond the specifically disclosed embodiments to other alternative embodiments and/or uses, and to modifications and equivalents thereof. Thus, the scope of the claims appended hereto is not limited by any of the particular embodiments described. For example, in any method or process disclosed herein, the acts or operations of the method or process can be performed in any suitable sequence and are not necessarily limited to any particular disclosed sequence. Various operations can be described as multiple discrete operations in turn, in a manner that can be helpful in understanding certain embodiments; however, the order of description should not be construed to imply that these operations are order dependent.

I claim:

1. A method for robotic active surgery, comprising the steps of: (a) get an image of a surgical site; (b) select in said image an object to track; and (d) accompanying a movement of said object with at least one active robotic instrument:
1.1. The method of claim 1, further comprising using said selection to scan every frame looking for said object.
1.2. The method of claim 1, further comprising improving tracking performance during tracking.
1.3. The method of claim 1, further comprising determining said object locations and changes of said object.
1.4. The method of claim 1, further comprising learning how said objects looks like and use that information to scan every frame searching for said object.
1.5. The method of claim 1, further comprising defining said object location in relation to at least one camera head.
1.6. The method of claim 1, further comprising defining a working distance of said camera head relative to said object.
1.7. The method of to claim 1, further comprising accompanying with at least one active camera head the movement of said object in space.
1.8. The method of claim 1, further comprising accompanying with at least one active robotic instrument said movement of said object in said surgery site.
1.9. The method of claim 1, further comprising transmitting said image to a screen.
1.10. The method of claim 1, further comprising observing in a screen said image of said moving object as if it is standing still.
1.11. The method of claim 1, further comprising, choosing in a toolbox a tip tool for said active robotic instrument inside said surgical site.
1.12. The method of claim 1, further comprising changing said robotic active instrument tip tool inside said surgical site.
1.13. The method of claim 1, further comprising using at least one active robotic instrument to perform said surgery.
1.14. The method of claim 1, further comprising sensing movement of said object and generating sensor output signals in response thereto;
1.15. The method of claim 1, further comprising calculating a demanded force signal for at least one actuator in response to said sensor output signals.
1.16. The method of claim 1, further comprising regulating a damped resistance to movement between said active robotic instrument and said object when said demanded force signal is within a predetermined range.

2. A method for a robotic active surgery, comprising the steps of:
a) selecting on a screen at least one point in a surgical site image;
b) choosing in a system database the best technique to perform said procedure; and
c) using an active robotic instrument to perform said procedure

3. A method for robotic active surgery, the method being characterized by:
a) detecting an event; and
b) moving a position of an ative robotic instrument in response to said detected event.
3.1. The method of claim 3, further comprising selectively switching the operating mode of said active robotic instrument between an active mode and a passive mode.
3.2. The method of claim 3, wherein said event comprises a user selection.
